(19) United States
(12) Patent Application Publication
(10) Pub. No.: US 2009/0131725 A1
(43) Pub. Date: May 21, 2009

(54) PROCESS FOR PRODUCING HYDROLYZATE

(75) Inventor: Kenichi Naka, Wakayama (JP)

Correspondence Address: OBLON, SPIVAK, MCCLELLAND MAIER & NEUSTADT, P.C., 1940 DUKE STREET, ALEXANDRIA, VA, 22314, US

(73) Assignee: KAO CORPORATION, Chuo-ku (JP)

(21) Appl. No.: 12/064,419

(22) PCT Filed: Aug. 21, 2006
(86) PCT No.: PCT/JP2006/316317

§ 371 Date: Feb. 21, 2008

(30) Foreign Application Priority Data
Aug. 22, 2005 (JP) 2005-240276

(51) Int. Cl. C07C 41/02

(52) U.S. Cl. 568/672

(57) ABSTRACT

In a method for producing a hydrolysate in which an organic compound and water are mixed and a hydrolysis reaction of the organic compound is performed, shear flow of the organic compound and the water at a shear rate U/Dmin of 5.5 sec⁻¹ or more (where Dmin is a flow channel minimum inner diameter (mm) in a mixing section and U is a flow rate (mm/sec) of a mixture of the organic compound and the water in the mixing section), thereby mixing the organic compound and the water, and the hydrolysis reaction of the organic compound is performed at a reaction temperature of 150° C. to 350° C. and a reaction pressure equal to or higher than a saturation vapor pressure of the water.

### TECHNICAL FIELD

The present invention relates to a method for producing a hydrolysate.

### BACKGROUND ART

Hydrolysis reaction such as ring-opening reaction of an organic compound and the like is industrially used in many situations. For example, glyceryl ether obtained by ring-opening reaction of glycidyl ether is a compound useful as a solvent, an emulsifier, a dispersant, a detergent, a foam booster and the like.

Although glyceryl ether is produced using a catalyst in general, as a method for producing glyceryl ether without using a catalyst, for example, a method in which glycidyl ether is brought into a hydrolysis reaction with subcritical water or the like has been known (see Patent Reference 1).

However, in the known method, depending on a mixture state of an organic compound and water, delay in reaction time, a side reaction of dimerizing the organic compound as a raw material and a generated hydrolysate are increased or like problems are caused.

Patent Reference 1: Japanese Laid-Open Publication No. 2002-88000.

### DISCLOSURE OF THE INVENTION

The present invention provides a method for effectively producing a high quality hydrolysate by making a mixture state of an organic compound and water be a good condition for reaction.

To achieve this, the present invention is directed to a method for producing a hydrolysate in which an organic compound and water are mixed and a hydrolysis reaction of the organic compound is performed. In the method, the mixing is performed under shear flow of the organic compound and the water at a shear rate U/Dmin of 5.5 sec⁻¹ or more (where Dmin is a flow channel minimum inner diameter (mm) in a mixing section and U is a flow rate (mm/sec) of a mixture of the organic compound and the water in the mixing section), and the hydrolysis reaction of the organic compound is performed at a reaction temperature of 150° C. to 350° C. and a reaction pressure equal to or higher than a saturation vapor pressure of the water.

### BRIEF DESCRIPTION OF THE DRAWINGS

FIG. 1 is a diagram schematically illustrating an exemplary device preferably used in an embodiment of a method for producing a hydrolysate according to the present invention.

FIG. 2 is a diagram schematically illustrating another exemplary device preferably used in an embodiment of a method for producing a hydrolysate according to the present invention.

### BEST MODE FOR CARRYING OUT THE INVENTION

The present invention is characterized in that when an organic compound and water are mixed to perform a hydrolysis reaction of the organic compound, under predetermined conditions, the organic compound and water are mixed and hydrolysis of the organic compound is performed.

According to the present invention, decomposition and a side reaction of an organic compound as a raw material can be suppressed in the hydrolysis reaction and thus deterioration of hue of the generated hydrolysate and the like can be prevented. Furthermore, according to the present invention, since the hydrolysis reaction is performed under a high temperature condition, the reaction can proceed with high selectivity even without using a catalyst, the step of removing a catalyst from a reactant is not necessary, and a high quality hydrolysate can be efficiently produced.

As long as the components do not inhibit achievement of desired effects of the present invention, components and the like described below can be used independently or two or more of the components can be combined and then used.

According to the present invention, an organic compound used as a raw material is not particularly limited as long as it is a compound which can be decomposed through hydrolysis. The hydrolysis reaction is preferably a ring-opening reaction using water. Accordingly, an organic compound used as a raw material is preferably a compound which has a ring structure and of which ring structure is opened due to the hydrolysis reaction. As such a compound, glycidyl ether expressed by the following general formula (I) is to preferable.

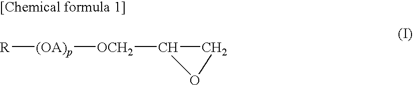

(where R is a hydrocarbon group in which part or all hydrogen atoms may be replaced with fluorine atoms, of which carbon number is 1 to 20 and which is saturated or unsaturated, OA is an oxyalkylene group which may be the same as or different from another OA and of which carbon number is 2 to 4, and p is a number of 0 through 20). Gryceryl ether obtained by ring-opening of glycidyl ether is a compound useful as a solvent, an emulsifier, a dispersant, a detergent, a foam booster and the like.

In the above formula, as the hydrocarbon group denoted by R, in which part or all hydrogen atoms may be replaced with fluorine atoms and of which carbon number is 1 to 20, for example, a straight-chain or branched-chain alkyl group of which carbon number is 1 to 20, a straight-chain or branched-chain alkenyl group of which carbon number is 2 to 20, an aryl group of which carbon number is 6 to 14 or the like may be used.

As the hydrocarbon group, specifically, a methyl group, an ethyl group, a n-propyl group, a n-butyl group, a n-pentyl group, a n-hexyl group, a n-heptyl group, a n-octyl group, a n-nonyl group, a n-decyl group, a n-dodecyl group, a tetradecyl group, a hexadecyl group, an octadecyl group, an eicosyl group, a 2-propyl group, a 2-butyl group, a 2-methyl-2-propyl group, a 2-pentyl group, a 3-pentyl group, a 2-hexyl group, a 3-hexyl group, a 2-octyl group, a 2-ethylhexyl group, a phenyl group, a benzyl group or the like can be used. Also, as the hydrocarbon group in which hydrogen atoms are replaced with fluorine atoms, for example, there are a perfluoroalkyl group such as a nanofluorohexyl group, a hexafluorohexyl group, a tridecafluorooctyl group, a heptadecafluorooctyl group, a heptadecafluorodecyl group and the like, obtained by replacing hydrogen atoms of the above-described hydrocarbon groups with fluorine atoms in an arbitrary manner without particular limits of the degree and location of replacement.

As specific examples of oxyalkylene group denoted by OA, of which carbon number is 2 to 4 are alkylene oxide such as an oxyethylene group, an oxytrimethylene group, an oxypropylene group, an oxybutylene group and the like.

Note that the carbon number of the hydrocarbon group denoted as R is preferably 1 to 12 in view of improving selectivity. Moreover, as for p, a number of 0 to 6 is preferable and 0 is more preferable.

According to the present invention, specifically, as glycidyl ether preferably used as a raw material, for example, n-butylglycidyl ether, 2-methyl-propylglycidyl ether, n-pentylglycidyl ether, 2-methyl-butylglycidyl ether, n-hexylglycidyl ether, 2-methyl-pentylglycidyl ether, phenylglycidyl ether, n-octylglycidyl ether, 2-ethyl-hexylglycidyl ether, isodecylglycidyl ether, n-stearylglycidyl ether and the like can be used.

According to the present invention, types of water used for the hydrolysis reaction of a raw material are not limited unless water inhibits achievement of desired effects of the present invention. As such water, for example, ion-exchange water, distilled water, reverse osmosis filtered water and the like can be used. Within the range in which nature of the present invention is not impaired, use of water containing salt and the like such as tap water is no problem.

An amount of water with respect to an organic compound is not particularly limited. However, in terms of molar conversion, it is preferably 20 to 500 times as large as a stoichiometric amount of water required for a reaction, more preferably 40 to 300 times as large as the stoichiometric amount thereof, and furthermore preferable 70 to 200 times as large as the stoichiometric amount thereof. In the above-described range, a side reaction such as dimerization of an organic compound as a raw material and a generated hydrolysate and the like can be suppressed and thus the selectivity of the hydrolysate can be further increased.

In the production method according to the present invention, the above-described organic compound as a raw material and water are mixed and then a hydrolysis reaction is performed.

According to the present invention, mixing of an organic compound and water and a hydrolysis reaction can be simultaneously performed in a reactor (Aspect 1) or an organic compound and water may be mixed in a mixer and then a hydrolysis reaction may be performed in a reactor (Aspect 2). In Aspect 1, the reactor serves as a mixing section and a reaction section, and in Aspect 2, the mixer corresponds to the mixing section and the reactor corresponds to the reaction section.

Mixing of an organic compound and water is performed at a shear rate (U/Dmin) of 5.5 (sec⁻¹) or more, preferably at a shear rate (U/Dmin) of 10 (sec⁻¹) or more, more preferably at a shear rate (U/Dmin) of 20 (sec⁻¹) or more, and further more preferably at a shear rate (U/Dmin) of 100 (sec⁻¹) or more. By performing shear flow of an organic compound and water to mix the organic compound and water, the organic compound and water can be reacted in a good mixed state through a hydrolysis reaction. Accordingly, in Aspect 2, it is preferable that after the mixture of the organic compound and water, the mixture is quickly brought to a hydrolysis reaction while keeping the mixed state as it is.

For shear rate U/Dmin, Dmin is a flow channel minimum inner diameter (mm) and U is a flow rate (mm/sec) of the mixture of the organic compound and water at Dmin. Assuming that the flow rate of the mixture is Q (ml/sec), U (mm/sec) can be obtained based on the following formula (1) where n is the number of the flow channel minimum inner diameter. For example, when a porous contraction flow type mixer is used, n is the number of pores in the mixer.

U (mm/sec)=Q×1000/(n×π×(Dmin)²/4) (1)

Moreover, a cross-sectional shape of the mixing portions does not have to be a circular shape. When the cross-sectional shape is other than a circular shape, U is a flow rate at a flow channel minimum cross-sectional area. In that case, a diameter of a circle having the same area as the flow channel minimum cross section is used as Dmin.

The flow channel minimum inner diameter of the mixing section is preferably 1 mm or more in view of productivity and is preferable 15 mm or less in view of achieving a good mixed state of an organic compound and water. In consideration of these views, the flow channel minimum inner diameter is preferably 1 to 15 mm and more preferably 1 to 10 mm. The flow channel minimum inner diameter of the mixing section means the flow channel minimum inner diameter of the mixer in Aspect 1 and also the flow channel minimum inner diameter of the mixer in Aspect 2.

A mixing time is not particularly limited as long as the mixing time is long enough to sufficiently mix an organic compound and water. In the case of a continuous type mixer, in general, the mixing time is preferably selected to be within the range from about 0.001 seconds to 10 hours. The range from about 0.001 seconds to 1 hour is more preferable and the range from 0.001 seconds to 10 minutes is further more preferable. The mixing time for a continuous type mixer means a time in which a reaction liquid is retained in the mixer and is indicated by a value obtained by dividing a volume of the mixer by a flow volume of reaction materials supplied to the reactor per unit time.

In view of increasing reactivity of an organic compound and water and in view of suppressing corrosion of a reactor, a reaction temperature for a hydrolysis reaction is 150° C. to 350° C., preferably 200° C. to 300° C. and more preferably 250° C. to 290° C. As for a reaction pressure, a hydrolysis reaction is performed under a condition where a pressure equal to or higher than a saturation vapor pressure of water is applied and water can be kept to be in a liquid state.

A reaction time varies depending on a reaction temperature, a type of a raw material to be used and the like and therefore can not be determined. However, in general, the reaction time is preferably selected to be within the range from 0.1 minute to 10 hours. The range from 0.1 minute to 1 hour is more preferable and the range from 0.1 minute to 10 minutes is further more preferable. In the case of a batch type reactor, the reaction time is counted from the completion of loading a raw material and the like. In the case of a continuous type reactor, the reaction time is counted from a timing at which a reaction has reached a stationary state. The reaction time of the continuous type reactor means a time in which a reaction liquid is retained in the reactor and is indicated by a value obtained by dividing a volume of the reactor by a flow volume of reaction materials supplied to the reactor per unit time.

According to the present invention, a hydrolysis reaction is preformed at a high temperature. Thus, the reaction proceeds even without a catalyst. However, an acid or alkali catalyst can be added. A catalyst used in the present invention is not particularly limited but, for example, an acid, a base or a combination of an acid and a base, which are in general used in hydrolysis reaction, can be used.

When a catalyst is used, a usage amount of the catalyst is not particularly limited as long as a desired reaction efficiency of a hydrolysis reaction of a raw material is achieved. However, in general, the usage amount is preferably 0.01 to 10 parts by weight and more preferably 0.1 to 5 parts by weight with respect to 100 parts by weight of an organic compound as a raw material.

As a mixer used for mixing an organic compound and water in advance, in the case of a batch type mixer, for example, a propeller mixer, an agihomo-mixer, a homo-mixer, a disk turbine paddle impeller having a high shear property, a pitched blade paddle impeller, a paddle blade impeller and the like can be preferably used. In the case of a continuous type mixer, for example, a pipe line mixer, a line homo-mixer, an ultrasonic mixer, a high pressure homogenizer, pumps such as a centrifugal pump having a high shear property, a disper mixer, a static mixer and like can be preferably used. Among the above-described examples, it is preferable to use a static mixer because it has a simple configuration and its maintenance can be done in a simple manner, compared to the other ones. Specifically, an orifice contraction flow type mixer is more preferable because a fluid shear rate at a mixing section thereof is high and high mixing effect can be achieved.

A temperature when an organic compound and water are mixed in a mixer is not particularly limited but is preferably about the same temperature as a reaction temperature.

As a reactor for performing a hydrolysis reaction, in the case of a continuous type reactor, a flow tube type reactor such as a tube type reactor, a tower type reactor, a semi-batch reactor such as a continuous stirred tank reactor and the like can be used.

In Aspect 1, the reactor serves as a mixing section and a reaction section. In this case, mixing is performed by flow and diffusion of a reaction liquid.

A material of the reactor used according to the present invention is not particularly limited. In general, a material used for chemical reaction can be arbitrarily used. Specific examples are metal materials such as steel, stainless steel, Fe—Cr—Ni alloy such as carpenter 20 and the like, copper alloy, aluminum alloy, Ni—Cr—Fe alloy, Ni—Cu alloy, Ni—Mo—Fe—Cr alloy, cobalt alloy, titanium alloy, zirconium alloy, molybdenum, chromium and the like, hard glass, silica glass, porcelain, glass lining, synthetic resin, ceramic materials and the like. Among the above-described materials, when a reaction takes place under a temperature condition close to a supercritical water condition where corrosion of materials is concerned, a metal material such as austenitic stainless steel, Ni—Cr—Fe alloy, Ni—Mo—Fe—Cr alloy and the like is preferable and Ni—Cr—Fe alloy and Ni—Mo—Fe—Cr alloy are more preferable.

According to the method of the present invention, a hydrolysis reaction can be performed by either one of a batch method in which a raw material at a required amount for 1 batch is supplied and a hydrolysis reaction for the amount is completed in a batch operation and a continuous method in which a raw material is continuously supplied and a hydrolysis reaction is performed. However, because a temperature can be increased/reduced in a short time, reaction conditions can be controlled in a simple manner and a reaction can be made to effectively proceed, it is preferable to continuously perform a hydrolysis reaction.

When a hydrolysis reaction is continuously performed, a tube type reactor is preferably used because it exhibits good operability and high resistance to pressure in a high pressure reaction. Furthermore, when a mixer is used, a static mixer is preferably used because it has a simple configuration and its maintenance can be performed in a simple manner. Specifically, an orifice contraction flow type mixer is more preferable because a fluid shear rate at a mixing section thereof is high and high mixing effect can be achieved.

After the completion of reaction, for example, a reacted mixture is cooled down to a desired temperature, evaporation or distillation, spontaneous sedimentation or centrifugal sedimentation or the like is performed as desired according to a known method to refine the mixture and separate the mixture from unreacted water, thus obtaining a hydrolysate.

### EXAMPLES

### Example 1

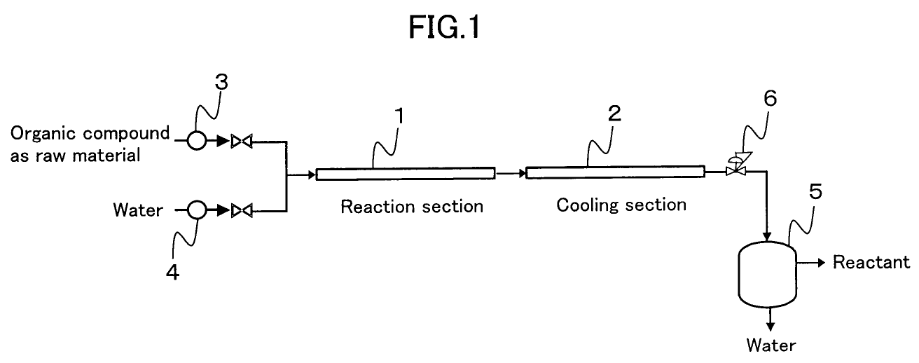

A reaction apparatus shown in FIG. 1 was used. The reaction apparatus of FIG. 1 includes a tube type reactor 1, a cooler 2, a raw material supply section 3, a water supply section 4 and a separate collection tank 5. Each of the raw material supply section 3 and the water supply section 4 is connected to the tube type reactor 1.

After preheating to the same temperature as a reaction temperature, 2-ethyl-hexylglycidyl ether as a raw material and ion exchange water were continuously supplied at 0.73 g/min and 7.03 g/min, respectively, from the raw material supply section 3 and water supply section 4 to the tube type reactor 1 (having a flow channel minimum inner diameter of 1.0 mm, a tube length of 10 m and being formed of SUS316).

In the tube type reactor 1, an inner fluid was heated so that a temperature (i.e., reaction temperature) of the inner fluid becomes 250° C., a pressure (i.e., reaction pressure) in the tube type reactor 1 was controlled by a back pressure valve 6 so as to be 5 MPa. Under the above-described temperature and pressure conditions, a hydrolysis reaction of an organic compound as a raw material was performed. The saturation vapor pressure of water at 250° C. was 4.0 MPa and the amount of water with respect to the organic compound as a raw material in a stationary state of the reaction was, in terms of molar conversion, 100 times as large as a stoichiometric amount of water required for the reaction.

After the hydrolysis reaction, a resultant mixture was cooled down to 40° C. to 50° C. in the cooler 2, and then the mixture was collected in the separate collection tank 5 through the back pressure valve 6. In the separate collection tank 5, the reacted mixture was split into layers and a reactant as an upper layer was collected.

The reactant was sampled at one hour after glycidyl ether was supplied and the hydrolysis reaction was started, and a reaction conversion ratio and a dimer selective ratio for glyceryl ether were obtained from a gas chromatogram (a gas chromatography apparatus: Agilent 6850 Series II manufactured by Agilent Technologies, a capillary column: HP-ULTRA2 having dimensions of 12 m×0.2 mm×0.33 μm, an internal standard substance: n-decan). Results are shown in Table 1. The reaction conversion ratio was calculated based on: reacted glycidyl ether (mol)/supplied glycidyl ether (mol)×100. The dimer production rate indicating a side reaction was calculated based on: a dimer amount (mol %) in the reactant/the reaction conversion ratio (mol %)×100.

### Example 2

The tube type reactor 1 having a tube length of 3.1 m was used, the reaction temperature was adjusted to be 270° C., the reaction pressure was adjusted to be 7 MPa, 2-ethyl-hexylglycidyl ether as a raw material was supplied at 0.23 g/min and ion exchange water was supplied at 2.2 g/min. Other than that, in the same manner as in Example 1, glyceryl ether was produced. Results are shown in Table 1. Note that a saturation vapor pressure of water at 270° C. is 5.5 MPa.

### Example 3

2-ethyl-hexylglycidyl ether as a raw material and ion exchange water were continuously supplied at 1.64 g/min and 15.9 g/min, respectively, to the tube type reactor 1 having a flow channel minimum inner diameter of 3.0 mm and a tube length of 2.0 m. Other than that, in the same manner as in Example 2, glyceryl ether was produced. Results are shown in Table 1.

### Example 4

The reaction temperature was adjusted to be 290° C. and the reaction pressure was adjusted to be 9 MPa. Other than that, in the same manner as in Example 3, glyceryl ether was produced. Results are shown in Table 1. Note that a saturation vapor pressure of water at 290° C. is 7.4 MPa.

### Example 5

2-ethyl-hexylglycidyl ether as a raw material and ion exchange water were continuously supplied at 18.17 g/min and 176 g/min, respectively, to the tube type reactor 1 having a flow channel minimum inner diameter of 10 mm and a tube length of 2.0 m. Other than that, in the same manner as in Example 4, glyceryl ether was produced. Results are shown in Table 1.

### Example 6

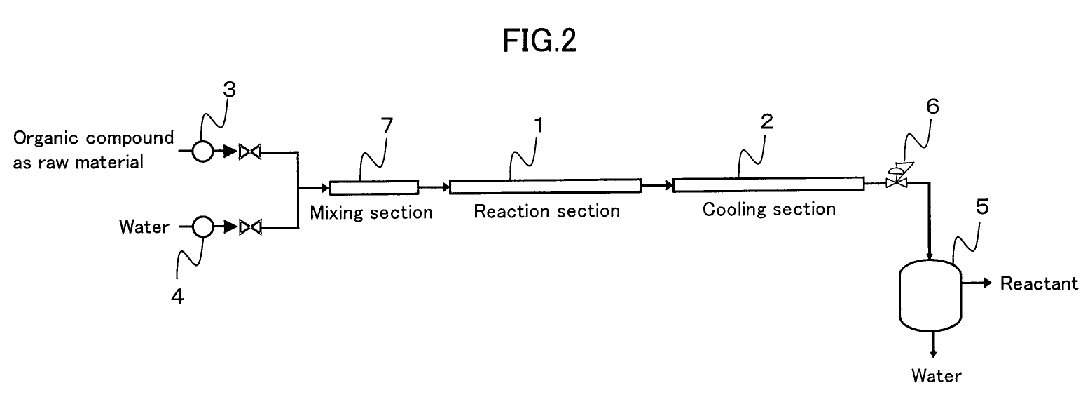

A reaction apparatus shown in FIG. 2 was used. The apparatus of FIG. 2 includes a tube type reactor 1, a cooler 2, a raw material supply section 3, a water supply section 4, a separate collection tank 5 and a mixer 7. Each of the raw material supply section 3 and the water supply section 4 is connected to the mixer 7.

After preheating to the same temperature as a reaction temperature, 2-ethyl-hexylglycidyl ether as a raw material and ion exchange water were continuously supplied at 67.9 g/min and 657 g/min, respectively, from the raw material supply section 3 and the water supply section 4 to the mixer 7. A contraction flow type mixer (in this case, Bunsan-kun manufactured by Fujikin, including 5 pairs of 4-pore block and 5-pore block and having a contraction section inner diameter of 1.0 mmφ and a pore length of 0.05 m) was provided in the mixer 7. Reaction materials mixed in the mixer 7 were continuously supplied to the tube type reactor 1 (having a flow channel inner diameter of 16 mm, a tube length of 5.4 m and being formed of SUS316).

In the tube type reactor 1, an inner fluid was heated so that a temperature (reaction temperature) of the inner fluid become 270° C. and a pressure (i.e., reaction pressure) in the tube type reactor 1 was adjusted to be 7 MPa. Under the temperature and pressure conditions, a hydrolysis reaction of an organic compound as a raw material was performed. In a stationary state of a reaction, an amount of water with respect to the organic compound as a raw material was, in terms of molar conversion, 100 times as large as a stoichiometric amount of water required for a reaction.

After the hydrolysis reaction, a mixture was cooled down to 40° C. to 50° C. in the cooler 2, and then the mixture was collected in the separate collection tank 5 through the back pressure valve 6. In the separate collection tank 5, the reacted mixture was split into layers and a reactant as an upper layer was collected.

The reactant was sampled at one hour after glycidyl ether was supplied and the hydrolysis reaction was started, and a reaction conversion ratio and a dimer selective ratio for glyceryl ether were obtained from the gas chromatogram. Results are shown in Table 1.

### Example 7

The reaction temperature was adjusted to be 290° C., the reaction pressure was adjusted to be 9 MPa and 2-ethyl-hexylglycidyl ether as a raw material and ion exchange water were continuously supplied at 102 g/min and 985 g/min, respectively, to the mixer 7. Other than that, in the same manner as in Example 6, glyceryl ether was produced. Results are shown in Table 1.

### Example 8

2-ethyl-hexylglycidyl ether as a raw material and ion exchange water were continuously supplied at 69.7 g/min and 472 g/min, respectively, to the mixer 7. In a stationary state of a reaction, an amount of water with respect to the organic compound as a raw material was, in terms of molar conversion, 70 times as large as a stoichiometric amount of water required for a reaction. Other than that, as in the same manner as in Example 6, glyceryl ether was produced. Results are shown in Table 1.

### Example 9

2-ethyl-hexylglycidyl ether as a raw material and ion exchange water were continuously supplied at 26.9 g/min and 520 g/min, respectively, to the mixer 7. In a stationary state of a reaction, an amount of water with respect to the organic compound as a raw material was, in terms of molar conversion, 200 times as large as a stoichiometric amount of water required for a reaction. Other than that, as in the same manner as in Example 8, glyceryl ether was produced. Results are shown in Table 1.

### Example 10

The tube type reactor 1 having a tube length of 10.8 m was used, isodecylglycidyl ether as a raw material and ion exchange water were supplied at 77.0 g/min and 647 g/min, respectively, to the mixer 7, the reaction temperature was adjusted to be 280° C. and the reaction pressure was adjusted to be 8 MPa. Other than that, in the same manner as in Example 6, glyceryl ether was produced. Results are shown in Table 1. Note that a saturation vapor pressure of water at 280° C. is 6.4 MPa.

### Example 11

The reaction apparatus of FIG. 2 was used. After preheating to the same temperature as a reaction temperature, 2-ethyl-hexylglycidyl ether as a raw material and ion exchange water were continuously supplied at 4650 g/min and 45000 g/min, respectively, to the mixer 7. A contraction flow type mixer (in this case, Bunsan-kun manufactured by Fujikin, including 5 pairs of 20-pore block and 25-pore block and having a contraction section inner diameter of 3.0 mmφ and a pore length of 0.05 m) was provided in the mixer 7. Reaction materials mixed in the mixer 7 were continuously supplied to the tube type reactor 1 (having a flow channel inner diameter of 50 mm and a tube length of 74 m).

In the tube type reactor 1, an inner fluid was heated so that the reaction temperature become 250° C. and the reaction pressure was adjusted to be 8 MPa. Under the temperature and pressure conditions, a hydrolysis reaction of an organic compound as a raw material was performed. Results are shown in Table 1.

### Comparative Example 1

Instead of the reaction apparatus of FIG. 2, the reaction apparatus of FIG. 1 was used. That is, the mixer 7 was not used. Other than that, in the same manner as in Example 6, glyceryl ether was produced. Results are shown in Table 1.

### Comparative Example 2

Instead of the reaction apparatus of FIG. 2, the reaction apparatus of FIG. 1 was used. That is, the mixer 7 was not used. Other than that, in the same manner as in Example 8, glyceryl ether was produced. Results are shown in Table 1.

### Comparative Example 3

Instead of the reaction apparatus of FIG. 2, the reaction apparatus of FIG. 1 was used. That is, the mixer 7 was not used. Other than that, in the same manner as in Example 9, glyceryl ether was produced. Results are shown in Table 1.

### Comparative Example 4

Instead of the reaction apparatus of FIG. 2, the reaction apparatus of FIG. 1 was used. That is, the mixer 7 was not used. Other than that, in the same manner as in Example 10, glyceryl ether was produced. Results are shown in Table 1.

In each of Examples 1 through 5 and Comparative Examples 1 through 4, a reactive apparatus which does not include the mixer 7 was used. However, the reactor 1 serves as a mixing section and a reaction section.

TABLE 1

| | | Example 1 | Example 2 | Example 3 | Example 4 | Example 5 | Example 6 | Example 7 | Example 8 |
|---|---|---|---|---|---|---|---|---|---|
| Organic compound as raw material* | | A | A | A | A | A | A | A | A |
| Flow rate of reaction materials | | | | | | | | | |
| Organic copound as raw material | (g/min) | 0.73 | 0.23 | 1.64 | 1.64 | 18.17 | 67.9 | 102 | 69.7 |
| | (ml/min) | 1.02 | 0.33 | 2.35 | 2.41 | 26.70 | 97.45 | 150 | 100 |
| Water | (g/min) | 7.03 | 2.20 | 15.9 | 15.9 | 176 | 657 | 985 | 472 |
| | (ml/min) | 9.17 | 3.01 | 21.7 | 22.9 | 254 | 898 | 1,420 | 645 |
| Mixer | | — | — | — | — | — | Contraction | Contraction | Contraction |
| | | | | | | | flow type | flow type | flow type |
| Flow channel minimum inner | (mm) | — | — | — | — | — | 1.0 | 1.0 | 1.0 |
| diameter Dmin | | | | | | | | | |
| Tube length | (m) | — | — | — | — | — | 0.05 | 0.05 | 0.05 |
| Residence time t | (min) | — | — | — | — | — | 0.11 | 0.071 | 0.14 |
| Reactor | | Tube type | Tube type | Tube type | Tube type | Tube type | Tube type | Tube type | Tube type |
| Flow channel minimum inner | (mm) | 1.0 | 1.0 | 3.0 | 3.0 | 10 | 16 | 16 | 16 |
| diameter Dmin | | | | | | | | | |
| Tube length | (m) | 10 | 3.1 | 2.0 | 2.0 | 2.0 | 5.4 | 5.4 | 5.4 |
| Residence time t | (min) | 1.0 | 1.0 | 0.8 | 0.8 | 0.8 | 1.5 | 1.0 | 2.0 |
| Mixer or reactor | | | | | | | | | |
| Flow channel minimum inner | (mm) | 1.0 | 1.0 | 3.0 | 3.0 | 10 | 1.0 | 1.0 | 1.0 |
| diameter Dmin | | | | | | | | | |
| The number n of flow channel | (—) | 1 | 1 | 1 | 1 | 1 | 4 | 4 | 4 |
| minimum inner diameter | | | | | | | | | |
| Flow rate U | (mm/sec) | 216 | 71 | 57 | 60 | 59 | 5,280 | 8,326 | 3,951 |
| U/Dmin | (1/sec) | 216 | 71 | 19 | 20 | 5.9 | 5,280 | 8,326 | 3,951 |
| Reaction conditions | | | | | | | | | |
| Reaction temperature | (° C.) | 250 | 270 | 270 | 290 | 290 | 270 | 290 | 270 |
| Reaction pressure | (MPa) | 5 | 7 | 7 | 9 | 9 | 7 | 9 | 7 |
| Mixing ratio of water/organic | (—) | 100 | 100 | 100 | 100 | 100 | 100 | 100 | 70 |
| compound as raw material | | | | | | | | | |
| (molar ratio) | | | | | | | | | |
| Reaction results | | | | | | | | | |
| Conversion ratio | (mol %) | 99.5 | 99.2 | 97.8 | 100.0 | 99.3 | 82.8 | 100.0 | 83.7 |
| Dimer amount in reactant | (mol %) | 1.4 | 1.2 | 2.0 | 2.2 | 2.1 | 1.8 | 1.6 | 2.1 |
| Dimer selective ratio | (mol %) | 1.4 | 1.3 | 2.1 | 2.2 | 2.1 | 2.1 | 1.6 | 2.5 |

| | | | | | Comparative | Comparative | Comparative | Comparative |
|---|---|---|---|---|---|---|---|---|
| | | Example 9 | Example 10 | Example 11 | Example 1 | Example 2 | Example 3 | Example 4 |
| Organic compound as raw material* | | A | B | A | A | A | A | B |
| Flow rate of reaction materials | | | | | | | | |
| Organic copound as raw material | (g/min) | 26.9 | 77.0 | 4,650 | 67.9 | 69.7 | 26.9 | 77.0 |
| | (ml/min) | 38.6 | 111 | 6,522 | 97.4 | 100 | 38.6 | 111 |
| Water | (g/min) | 520 | 647 | 45,000 | 657 | 472 | 520 | 647 |
| | (ml/min) | 711 | 907 | 58,693 | 898 | 645 | 711 | 907 |
| Mixer | | Contraction | Contraction | Contraction | — | — | — | — |
| | | flow type | flow type | flow type | | | | |
| Flow channel minimum inner | (mm) | 1.0 | 1.0 | 3.0 | — | — | — | — |
| diameter Dmin | | | | | | | | |
| Tube length | (m) | 0.05 | 0.05 | 0.05 | — | — | — | — |
| Residence time t | (min) | 0.14 | 0.11 | 0.013 | — | — | — | — |
| Reactor | | Tube Type | Tube type | Tube type | Tube type | Tube type | Tube Type | Tube type |
| Flow channel minimum inner | (mm) | 16 | 16 | 50 | 16 | 16 | 16 | 16 |
| diameter Dmin | | | | | | | | |
| Tube length | (m) | 5.4 | 10.8 | 74 | 5.4 | 5.4 | 5.4 | 10.8 |
| Residence time t | (min) | 2.0 | 3.0 | 3.0 | 1.5 | 2.0 | 2.0 | 3.0 |
| Mixer or reactor | | | | | | | | |
| Flow channel minimum inner | (mm) | 1.0 | 1.0 | 3.0 | 16 | 16 | 16 | 16 |
| diameter Dmin | | | | | | | | |
| The number n of flow channel | (—) | 4 | 4 | 20 | 1 | 1 | 1 | 1 |
| minimum inner diameter | | | | | | | | |
| Flow rate U | (mm/sec) | 3,974 | 5,405 | 7,689 | 83 | 63 | 64 | 83 |
| U/Dmin | (1/sec) | 3,974 | 5,405 | 2,563 | 5.2 | 4.0 | 4.0 | 5.2 |
| Reaction conditions | | | | | | | | |
| Reaction temperature | (° C.) | 270 | 280 | 250 | 270 | 270 | 270 | 280 |
| Reaction pressure | (MPa) | 7 | 8 | 8 | 7 | 7 | 7 | 8 |
| Mixing ratio of water/organic | (—) | 200 | 100 | 100 | 100 | 70 | 200 | 100 |
| compound as raw material | | | | | | | | |
| (molar ratio) | | | | | | | | |
| Reaction results | | | | | | | | |
| Conversion ratio | (mol %) | 99.1 | 96.5 | 99.6 | 60.3 | 66.8 | 76.7 | 94.9 |
| Dimer amount in reactant | (mol %) | 1.7 | 3.8 | 3.2 | 1.2 | 1.7 | 1.2 | 5.1 |
| Dimer selective ratio | (mol %) | 1.7 | 3.9 | 3.2 | 1.9 | 2.6 | 1.6 | 5.4 |

*Organic compound as raw material A: 2-ethyl-hexylglycidyl ether Organic compound as raw material B: isodecylglycidyl ether

According to the results described above, compared to the comparative examples, in each of the Examples, the conversion ratio is high and the dimer selective ratio is low. This shows that a hydrolysis reaction was efficiently performed.

### INDUSTRIAL APPLICABILITY

As has been described, the present invention is useful as a method for producing a hydrolysate, in which an organic compound and water are mixed and a hydrolysis reaction of the organic compound is performed. A hydrolysate obtained according to the present invention, e.g., glyceryl ether obtained by hydrolysis of glycidyl ether can be used as a solvent, an emulsifier, a dispersant, a detergent, a foam booster and the like.

1. A method for producing a hydrolysate in which an organic compound and water are mixed and a hydrolysis reaction of the organic compound is performed,
wherein the mixing is performed under shear flow of the organic compound and the water at a shear rate U/Dmin of 5.5 sec⁻¹ or more (where Dmin is a flow channel minimum inner diameter (mm) in a mixing section and U is a flow rate (mm/sec) of a mixture of the organic compound and the water in the mixing section), and
the hydrolysis reaction of the organic compound is performed at a reaction temperature of 150° C. to 350° C. and a reaction pressure equal to or higher than a saturation vapor pressure of the water.

2. The method of claim 1, wherein the hydrolysis reaction of the organic compound is performed without using a catalyst.

3. The method of claim 1, wherein the mixing of the organic compound and the water and the hydrolysis reaction of the organic compound are simultaneously performed.

4. The method of claim 1, wherein after mixing the organic compound and the water, the hydrolysis reaction of the organic compound is subsequently performed.

5. The method of claim 1, wherein the hydrolysis reaction of the organic compound is a ring-opening reaction by the water.

6. The method of claim 1, wherein the organic compound is glycidyl ether expressed by a general formula (1) 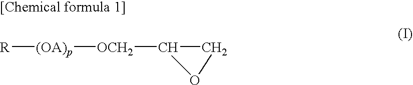
(where R is a hydrocarbon group in which part or all hydrogen atoms may be replaced with fluorine atoms, of which carbon number is 1 to 20 and which is saturated or unsaturated, OA is an oxyalkylene group which may be the same as or different from another OA and of which carbon number is 2 to 4, and p is a number of 0 to 20).

7. The method of claim 1, wherein an amount of the water with respect to the organic compound is, in terms of molar conversion, 20 to 500 times as large as a stoichiometric amount of water required for the reaction.

8. The method of claim 1, wherein the hydrolysis reaction of the organic compound is continuously performed.

9. The method of claim 1, wherein the flow channel minimum inner diameter in the mixing section in which the organic compound and the water are mixed is 1 mm to 15 mm.

10. The method of claim 1, wherein the organic compound and the water are mixed by flowing the organic compound and the water through a static mixer.

11. The method of claim 10, wherein the static mixer is a contraction flow type mixer.
